(19) United States
(12) Patent Application Publication
(10) Pub. No.: US 2025/0375372 A1
(43) Pub. Date: Dec. 11, 2025

(54) COSMETIC COMPOSITION INCLUDING AN ALOE-TREATED COLORANT

(71) Applicant: L'OREAL, PARIS (FR)

(72) Inventors: Jody EBANKS, CLARK, NJ (US); Tsang-Min HUANG, SCOTCH PLAINS, NJ (US)

(73) Assignee: L'OREAL, PARIS (FR)

(21) Appl. No.: 18/734,661

(22) Filed: Jun. 5, 2024

(51) Int. Cl. A61K 8/9794; A61Q 1/02; A61Q 1/06; A61Q 3/02

(52) U.S. Cl. CPC A61K 8/9794; A61Q 1/02; A61Q 1/06; A61Q 3/02; A61K 2800/43

(57) ABSTRACT

A cosmetic composition is provided. The cosmetic composition includes a continuous fatty phase including one or more fatty substances. It further includes a colorant dispersed in the fatty phase. The colorant includes a substrate; at least one dye; and a surface treatment. The surface treatment includes aloe. Methods are also provided.

### TECHNICAL FIELD

The present disclosure is drawn to the field of cosmetic compositions, and specifically, to cosmetic compositions including colorant and having reduced color shift.

### BACKGROUND

Cosmetic formulations including colorant, particularly colorants that include dye may be susceptible to shifting appearance of the color over time, particularly in compositions that are liquid. The present inventors have found this to be the case with cosmetic compositions having a continuous fatty phase. This can be problematic as consumers typically desire products that will have a predictable color appearance on keratinous substrates to which it is applied (e.g., skin, lips, nails, and the like). Accordingly, a need exists for improved color stability for cosmetic systems.

### BRIEF SUMMARY

A cosmetic composition including a continuous fatty phase that includes one or more fatty substances and a colorant dispersed in the fatty phase is provided. The colorant includes a substrate, at least one dye, and a surface treatment including aloe. The substrate may include at least one xanthene dye, such as disodium 2′,4′,5′, 7′-tetrabromo-4,5,6,7-tetrachlorofluorescein. The substrate may include at least one metal salt such as an aluminum salt.

The colorant further includes a surface treatment that includes aloe. The aloe of the surface treatment may be present in a concentration by weight in the composition that is less than the concentration by weight of the substrate in the cosmetic composition.

The surface treatment may be a hydrophobic surface treatment and may further include one or more vegetable fatty compounds, such as a coconut oil.

The colorant may be present in the composition in a concentration by weight that is from about 0.01% to about 10%.

The cosmetic composition is, in certain embodiments, substantially free of water. In certain embodiments the cosmetic composition is liquid. In certain embodiments the composition includes an internal polyol phase within an external fatty phase. In certain other embodiments the composition further includes a fatty acid ester of a polyol. In certain other embodiments the composition further includes one or more film-forming polymers. In certain embodiments, the composition is substantially free of C1-C4 monoalcohols and C1-C4 monoacetates. In certain embodiments, the compostion further includes a UV-stabilizing compound such as one selected from piperidinols, benzotriazoles, and combinations thereof. According to certain other embodiments, the composition includes at least about 35% by weight of fatty substances. According to certain other embodiments, the composition exhibits at least a 35% improvement in color change reduction (such as from 40% improvement to 70% improvement), where % Improvement=(ΔE_{u,ns}−ΔEₛₐₘₚₗₑ)/ΔE_{u,ns}, versus an identical composition except with (1) the colorant having no surface treatment, and (2) having no UV-stabilizers, when tested according to methods described herein.

According to certain other embodiments, the composition includes a plurality of or even all of the limitations specified above.

According to another aspect of the invention, a method of the above-described cosmetic compositions includes providing the cosmetic composition and applying the cosmetic composition to a lip, skin, or nails, such as for example to provide a cosmetically enhanced appearance. Application to the lips is particularly notable.

### DETAILED DESCRIPTION

As used herein, articles such as “a” and “an” as used herein, are understood to mean one or more of what is claimed or described.

As used herein, the expression “at least one” means one or more and thus includes individual components as well as mixtures/combinations.

All percentages, parts and ratios are based upon the total weight of the compositions of the present invention unless otherwise indicated. Other than in the operating examples, or where otherwise indicated, all numbers expressing quantities (e.g., concentrations, ratios, and the like) of ingredients and/or reaction conditions are to be understood as being modified in certain embodiments by the term “about,” meaning within 10% to 15% of the indicated number (e.g. “about 10%” means 8.5% to 11.5% such as 9% to 11%, and “about 2%” means from 1.7% to 2.3 such as from 1.8% to 2.2%).

Similarly, for ratios, the modifier “about” means within 10% or 15% of the ratio. For example, about 4:1 means from 3.4:1 to 4.6:1, preferably 3.6:1 to 4.4:1. As readily understood by one skilled in the art, where the first ingredient in a ratio is less than the second, then a ratio may be expressed “inversely.” For example, if a second ingredient, B is present in an amount or concentration that is 2.5 times greater than that of ingredient A, this may be identified as an A: B ratio of 1:2.5. “About 1:10,” means from 1:8.5 to 1:11.5, preferably 1:9 to 1:11. As one skilled in the art will understand, the modifier “about” also encompasses the exact number or ratio specified. Unless otherwise indicated, all concentrations shown as percentages are concentrations by weight and also, unless otherwise indicated, relate to the entire composition as a whole.

As used herein, the term “between” two number is intended to be inclusive of the bounding numbers. Thus, between 0 and 1 would include both 0 and 1.

Numerical ranges, unless specifically stated otherwise, are inclusive of endpoints and meant to include all combinations and sub-combinations. Notably, from about 5%, 10% or 15% to about 20%, 50% or 60% may refer to about 5% to about 20%, about 5% to about 50%, about 5% to about 60%, about 10% to about 20%, about 10% to about 50%, about 10% to about 60%, about 15% to about 20%, about 15% to about 50%, or about 15% to about 60%. Further as used herein a range of ratios is meant to include every specific ratio within, and combination of subranges between the given ranges.

As used herein, the terms “include”, “includes” and “including” are meant to be non-limiting.

As used herein, the term “substantially free (of an ingredient)” means that the composition contains less than 1%, such as less than 0.5% of the identified ingredient, such as less than 0.3% of the identified ingredient. The term “essentially free (of an ingredient)” means the composition contains less than 0.25%, such as less than 0.1% of the identified ingredient, preferably below detectable amounts of the identified ingredient, and most preferably none of the identified ingredient.

The term “anhydrous”, as used herein, refers to a composition that is substantially free of or essentially free of water.

The disclosed compositions surprisingly provide improved color stability as they are less susceptible to color changes than compositions of the prior art. When used according to certain embodiments, in a composition having an internal phase including polyol such as a polyol-in oil emulsion, the compositions can further provide a low tack sensory feel.

Thus, a cosmetic composition may be provided. The composition may be a liquid (e.g., takes the shape of its container). In certain embodiments the composition is an emulsion.

### Fatty Phase and Fatty Substances

According to preferred embodiments of the present invention, compositions include a fatty phase that includes one or more fatty substances. The fatty phase may optionally comprise dispersed particulate materials. The fatty phase may be a continuous fatty phase such as a continuous external phase of an emulsion or a continuous fatty phase with no additional liquid phases present in the composition.

Suitable fatty substances include oil(s), wax(es) and other fatty compounds. “Oil” means any non-aqueous medium which is liquid at ambient temperature (25° C.) and atmospheric pressure (760 mm Hg). A “wax” for the purposes of the present disclosure is a lipophilic fatty compound that is solid at ambient temperature (25° C.) and changes from the solid to the liquid state reversibly, having a melting temperature of more than 30° C. and, for example, more than 45° C., which can be as high as 150° C., a hardness of more than 0.5 MPa at ambient temperature, and an anisotropic crystalline organization in the solid state. By taking the wax to its melting temperature, it is possible to use wax(es) by themselves as carriers and/or it is possible to make wax(es) miscible with the oils to form a microscopically homogeneous mixture.

Suitable oils include volatile and/or non-volatile oils. Such oils can be any acceptable oil including but not limited to silicone oils and/or hydrocarbon oils. “Hydrocarbon (based) oils,” as differentiated from silicones, may in certain embodiments include only hydrogen, carbon and optional oxygen atoms, but include no silicon atoms. In other embodiments, atoms other than silicon (e.g., halogens, nitrogen, phosphorus) may also additionally be included.

The volatility of the solvents/oils can be determined using the evaporation speed as set forth in U.S. Pat. No. 6,338,839, the contents of which are incorporated by reference herein. According to certain embodiments of the present invention, the composition comprises at least one non-volatile oil.

Examples of non-volatile oils that may be used in the present invention include, but are not limited to, polar oils such as:
hydrocarbon-based plant oils with a high triglyceride content consisting of fatty acid esters of glycerol, the fatty acids of which may have varied chain lengths, these chains possibly being linear or branched, and saturated or unsaturated; these oils are especially wheat germ oil, corn oil, sunflower oil, karite butter, castor oil, sweet almond oil, macadamia oil and the like. Other triglyceride such as or caprylic/capric acid triglycerides are also suitable;
synthetic oils or esters of formula R₅COOR₆ in which Rs represents a linear or branched higher fatty acid residue containing from 1 to 40 carbon atoms, including from 7 to 19 carbon atoms, and R₆ represents a branched hydrocarbon-based chain containing from 1 to 40 carbon atoms, including from 3 to 20 carbon atoms, with R₆+R₇≥10, such as, for example, Purcellin oil (cetostearyl octanoate), isononyl isononanoate, octyldodecyl neopentanoate, C₁₂ to C₁₅ alkyl benzoate, isopropyl myristate, 2-ethylhexyl palmitate, and octanoates, decanoates or ricinoleates of alcohols or of polyalcohols; hydroxylated esters, for instance isostearyl lactate or diisostearyl malate; and pentaerythritol esters; and various synthetic ethers containing from 10 to 40 carbon atoms; C₈ to C₂₆ fatty alcohols, for instance oleyl alcohol, cetyl alcohol, stearyl alcohol, and cetearyl alcohol; and mixtures thereof.

Further, examples of non-volatile oils that may be used in the present invention include, but are not limited to, non-polar, non-silicone oils/hydrocarbon oils and/or waxes such as linear or branched hydrocarbons of mineral, animal or synthetic origin which may have more than 16 carbon atoms. Non-limiting examples may include liquid paraffins, liquid petroleum jell, and squalane—and in other embodiments such oils may also include liquid or solid polyolefins, in particular petrolatum, paraffin oil, squalene, hydrogenated polyisobutene, hydrogenated polydecene, mineral oil, pentahydrosqualene, and mixtures thereof.

Further, examples of non-volatile oils that may be used in the present invention include, but are not limited to, silicone oils such as dimethicone (polydimethylsiloxane) of various viscosities as well as phenylated silicone oils such as phenyl trimethicone and trimethylsiloxyphenyl dimethicone.

According to preferred embodiments, if present, the at least one oil is present in the compositions of the present invention in an amount ranging from about 5%, 10%, 15%, or 20% to about 30%, 50%, 60% or 80% by weight, more preferably from about 10% to about 70% by weight, and most preferably from about 15 to about 60% by weight, based on the total weight of the composition, including all ranges and subranges within these ranges.

According to preferred embodiments of the present invention, the compositions of the present invention further comprise at least one wax. Suitable examples of waxes that can be used in accordance with the present disclosure include those generally used in the cosmetics field: they include but are not limited to those of natural origin, such as beeswax, carnauba wax, candelilla wax, paraffin wax, lignite wax or microcrystalline wax, ceresin or ozokerite, and hydrogenated oils such as hydrogenated castor oil or jojoba oil; synthetic waxes such as the polyethylene waxes obtained from the polymerization or copolymerization of ethylene, and Fischer-Tropsch waxes, or else esters of fatty acids, such as octacosanyl stearate, glycerides which are concrete at 30° C., for example at 45° C.

According to certain embodiments of the present invention, the compositions of the present invention further include at least one silicone wax. Examples of suitable silicone waxes include, but are not limited to, silicone waxes such as alkyl- or alkoxydimethicones having an alkyl or alkoxy chain ranging from 10 to 45 carbon atoms, poly (di) methylsiloxane esters which are solid at 30° C. and whose ester chain comprising at least 10 carbon atoms, di(1,1,1-trimethylolpropane) tetrastearate, which is sold or manufactured by Heterene under the name HEST 2T-4S; alkylated silicone acrylate copolymer waxes comprising at least 40 mole % of siloxy units having the formula (R₂R′SiO_{1/2})ₓ(R″SiO_{3/2})_{y}, where x and y have a value of 0.05 to 0.95, R is an alkyl group having from 1 to 8 carbon atoms, an aryl group, a carbinol group, or an amino group, R is a monovalent hydrocarbon having 9-40 carbon atoms, R″ is a monovalent hydrocarbon group having 1 to 8 carbon atoms, an aryl group such as those disclosed in U.S. patent application 2007/0149703, the entire contents of which is hereby incorporated by reference, with a particular example being C30-C45 alkyldimethylsilyl polypropylsilsesquioxane; and mixtures thereof.

According to preferred embodiments, the compositions of the present invention contain less than 5% wax. According to preferred embodiments, the compositions of the present invention contain less than 4% wax.

In other embodiments, if present, the wax or waxes may be present in an amount ranging from about 0.25% or 0.5% to about 1%, 2%, 3%, 5% by weight relative to the total weight of the composition, including all ranges and subranges therebetween.

According to certain embodiments, the compositions of the present invention preferably comprise one or more volatile silicone oils. Examples of such volatile silicone oils include linear or cyclic silicone oils having a viscosity at room temperature less than or equal to 6cSt and having from 2 to 7 silicon atoms, these silicones being optionally substituted with alkyl or alkoxy groups of 1 to 10 carbon atoms. Specific oils that may be used in the invention include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane, PDMS DC 200 (2cSt) from Dow Corning, and their mixtures.

Further, a volatile linear silicone oil may be employed in the present invention. Suitable volatile linear silicone oils include those described in U.S. Pat. No. 6,338,839 and WO03/042221, the contents of which are incorporated herein by reference. In one embodiment the volatile linear silicone oil is decamethyltetrasiloxane. In another embodiment, the decamethyltetrasiloxane is further combined with another solvent that is more volatile than decamethyltetrasiloxane.

According to certain embodiments of the present invention, the composition of preferably comprises one or more volatile non-silicone oils and may be selected from volatile hydrocarbon oils, volatile esters and volatile ethers. Examples of such volatile non-silicone oils include, but are not limited to, volatile hydrocarbon oils having from 8 to 16 carbon atoms and their mixtures and in particular branched C₈ to C₁₆ alkanes such as C₈ to C₁₆ isoalkanes (also known as isoparaffins), isohexacecane, isododecane, isodecane, and for example, the oils sold under the trade names of Isopar or Permethyl. Preferably, the volatile non-silicone oils have a flash point of at least 40° C.

The one or more fatty substances may also include particular other classifications of fatty compounds—which may be volatile or non-volatile; silicone, or hydrocarbon. For example. In some embodiments, the fatty substances may include a dilinoleate ester dimer. Non-limiting examples of such dilinoleate ester dimers include: bis-behenyl/isostearyl/phytosteryl dimer dilinoleyl dimer dilinoleate; phytosteryl isostearyl dimer dilinoleate; phytosteryl dilinoleate/isostearyl dimer dilinoleate cetyl/stearyl/behenyl; and/or diisostearoyl polyglyceryl-3 dimer dilinoleate.

In some embodiments, the fatty substances may include a triester containing at least 35 carbon atoms, such as between 35 and 70 carbon atoms in total. Non-limiting examples of such triesters include triesters of a tricarboxylic acid, such as triisostearyl citrate, or tridecyl trimellitate, or glycol triesters of monocarboxylic acids such as polyglyceryl-2 triisostearate. In a preferred embodiment, the triester is tridecyl trimellitate.

In some embodiments, the fatty substances may include a triglycerides of a saturated or unsaturated C₈-C₂₀ fatty acid. Non-limiting examples include caprylic/capric triglyceride, glyceryl triheptanoate, glyceryl trioctanoate, or glyceryl triisostearate.

In some embodiments, the fatty substances may include a plant-derived oil. Non-limiting examples include Zea mays (corn) oil, brassica campestris (rapeseed) seed oil, butyrospermum parkii (shea) butter, prunus amygdalus dulcis (sweet almond) oil, prunus armeniaca (apricot) kernel oil, and Helianthus annuus (sunflower) seed oil, Olea europaea (olive) oil, Cocos nucifera (coconut) oil, and combinations thereof.

In some embodiments, the fatty substances may include a monoester of a monoalcohol. Non-limiting examples include dihydroabietyl behenate; octyldodecyl behenate; isocetyl behenate; isostearyl lactate; lauryl lactate; linoleyl lactate; oleyl lactate; and the like.

In some embodiments, the fatty substances may include a pentaerythritol ester, such as pentaerythrityl tetraisostearate.

In some embodiments, the fatty substances may include an alkylene carbonate. Non-limiting examples include C₄-C₆ cyclic carbonates, particularly propylene carbonate.

In some embodiments, the fatty substances may include one or more fatty acid esters of glycerol or polyglycerol. The fatty acid ester(s) of polyglycerol may be esters resulting from the reaction of polyglycerol comprising from 2 to 12 glycerol units such as from 10 to 18 carbon atoms. Non-limiting examples of fatty acid esters of glycerol or polyglycerol include: polyglyceryl-2 distearate; polyglyceryl-10 decastearate; glyceryl oleate; glyceryl stearate; polyglyceryl-5 hexastearate; and the like.

The total amount of fatty substance(s) may be present in a total amount of at least about 30% by weight of the cosmetic composition. The fatty substance(s) may be present in a total amount of at least 35%, such as at least 40% by weight of the cosmetic composition, such as at least 45%, such as at least 50%, such as at least 55% by weight of the cosmetic composition. According to other embodiments, the fatty substance(s) may be present in a total concentration from about 35%, 40%, 45% or 50% to about 50%, 60%, 70%, 80%, 90% or 95% by weight.

### Water

Water may, in certain embodiments may be included such as in an emulsified or stabilized internal phase. The composition is however, in certain embodiments, substantially free of water. In certain embodiments, the composition is essentially free of water.

### Colorant

The composition includes one or more colorants dispersed in the fatty phase. The colorant(s) includes a substrate, one or more dyes, and a surface treatment. The colorant is intended to color and/or opacify the composition and preferably the deposited layer produced with the composition.

The substrate may be formed from or include one or more materials that are insoluble in the fatty phase. The substrate may include for example forms or salts of metals, such as salts of sodium, potassium, calcium, barium, aluminum, zirconium, strontium or titanium that are insoluble in the fatty phase(s) and, in certain embodiments, insoluble or sparingly soluble in water as well. Aluminum salts are particularly notable. Examples of aluminum salts include alumina (e.g. alumina hydrate). Substrates formed from or including organic materials such as rosin as also useful. Substrates formed from or including mixed organic/inorganic materials such as aluminum benzoate are also useful. The colorant may comprise from about 40% to about 90% by weight of substrate.

The colorant generally includes one or more dyes that may be formed, treated or coated on the substrate. Accordingly, the colorant may be susceptible to the dye experiencing instability in its visual appearance over time as it is exposed to one or more of: changes or elevations in temperature, exposure to light or other radiation, exposure to fluid components in the composition.

Suitable dyes used in the colorant include, for example, salts of acid dyes, such as azo, anthraquinone, indigoid, xanthene, pyrene, quinoline, triphenylmethane or fluoran dyes, these dyes possibly comprising at least one carboxylic or sulfonic acid group. In other embodiments the dyes(s) are selected from azo dyes, anthraquinone dyes, indigoid dyes, xanthene dyes, pyrene dyes, quinoline dyes, triphenylmethane dyes or fluoran dyes.

In notable embodiments, the dye is a xanthene dye. In certain embodiments, the substrate includes a metal salt and the xanthene dye.

In another notable embodiments, the xanthene dye is disodium 2′,4′,5′,7′-tetrabromo-4,5,6,7-tetrachlorofluorescein.

The colorant may comprise from about 5% or 10% to about 35% or 40% by weight of the one or more dyes.

Dyes suitable for use in the present invention may include for example D&C Blue No. 4, D&C Brown No. 1, D&C Green No. 5, D&C Green No. 6, D&C Orange No. 4, D&C Orange No. 5, D&C Orange No. 10, D&C Orange No. 11, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, D&C Violet No. 2, D&C Yellow No. 7, D&C Yellow No. 8, D&C Yellow No. 10, D&C Yellow No. 11, FD&C Blue No. 1, FD&C Green No. 3, FD&C Red No. 40, FD&C Yellow No. 5 or FD&C Yellow No. 6. Red 28 is particularly notable.

The dye may be provided or formed on one of the substrates described herein. As such the substrate/dye may be a so-called “lake pigment.” Examples of suitable lake pigments include D&C Red No. 2 Aluminum lake, D&C Red No. 3 Aluminum lake, D&C Red No. 4 Aluminum lake, D&C Red No. 6 Aluminum lake, D&C Red No. 6 Barium lake, D&C Red No. 6 Barium/Strontium lake, D&C Red No. 6 Strontium lake, D&C Red No. 6 Potassium lake, D&C Red No. 7 Aluminum lake, D&C Red No. 7 Barium lake, D&C Red No. 7 Calcium lake, D&C Red No. 7 Calcium/Strontium lake, D&C Red No. 7 Zirconium lake, D&C Red No. 8 Sodium lake, D&C Red No. 9 Aluminum lake, D&C Red No. 9 Barium lake, D&C Red No. 9 Barium/Strontium lake, D&C Red No. 9 Zirconium lake, D&C Red No. 10 Sodium lake, D&C Red No. 19 Aluminum lake, D&C Red No. 19 Barium lake, D&C Red No. 19 Zirconium lake, D&C Red No. 21 Aluminum lake, D&C Red No. 21 Zirconium lake, D&C Red No. 22 Aluminum lake, D&C Red No. 27 Aluminum lake, D&C Red No. 27 Aluminum/Titanium/Zirconium lake, D&C Red No. 27 Barium lake, D&C Red No. 27 Calcium lake, D&C Red No. 27 Zirconium lake, D&C Red No. 28 Aluminum lake, D&C Red No. 30 lake, D&C Red No. 31 Calcium lake, D&C Red No. 33 Aluminum lake, D&C Red No. 34 Calcium lake, D&C Red No. 36 lake, D&C Red No. 40 Aluminum lake, D&C Blue No. 1 Aluminum lake, D&C Green No. 3 Aluminum lake, D&C Orange No. 4 Aluminum lake, D&C Orange No. 5 Aluminum lake, D&C Orange No. 5 Zirconium lake, D&C Orange No. 10 Aluminum lake, D&C Orange No. 17 Barium lake, D&C Yellow No. 5 Aluminum lake, D&C Yellow No. 5 Zirconium lake, D&C Yellow No. 6 Aluminum lake, D&C Yellow No. 7 Zirconium lake, D&C Yellow No. 10 Aluminum lake, FD&C Blue No. 1 Aluminum lake, FD&C Red No. 4 Aluminum lake, FD&C Red No. 40 Aluminum lake, FD&C Yellow No. 5 Aluminum lake or FD&C Yellow No. 6 Aluminum lake. Red 28 Lake is particularly notable, such as SUNCROMA D&C RED 28 AL LAKE C14-6623 available from Sun Chemical.

To form a colorant suitable for use in the present invention, any of various pigments formed from a substrate such as those described herein and treated with a dye, such as those also described herein may be then surface treated with a surface treatment including aloe.

“Aloe” refers to an extract from an aloe plant such as Aloe vera. The extract may be from an inner leaf (gel) extract, a whole leaf extract or a dried latex extract. In certain embodiments the colorant includes an Aloe vera leaf extract.

The surface treatment may be at least partially formed over the substrate and the dye. The surface treatment may be a hydrophobic surface treatment and in certain embodiments the hydrophobic surface treatment further includes one or more vegetable fatty compounds, e.g., coconut oil or other vegetable oils. The colorant may comprise from about 0.5% or 1% to about 2%, 5% or 10% by weight of the surface treatment.

According to certain embodiments, when considering the composition as a whole, the aloe and the substrate may each be present in a respective concentration by weight wherein the concentration by weight of the substrate is greater than the concentration by weight of the aloe.

The colorant with the hydrophobic surface treatment comprising aloe may be present in the composition in a concentration from about 0.01% to about 10% by weight. According to certain embodiments, the colorant is present in a concentration from about 0.01%, 0.05%, 0.1% or 0.25% to about 0.25%, 0.5%, 1%, 2% 5% or 10% by weight in the composition. In certain embodiments the total amount of all colorants fall within one or more of the above listed ranges.

According to certain embodiments, when considering the composition as a whole, the aloe and the substrate may each be present in a respective concentration by weight wherein the concentration by weight of the substrate is greater than the concentration by weight of the aloe.

Additional colorants such as “pearlescent agents,” which exhibit a color effect via optical interference; as well as other color pigments including, for example oxides of iron may also be included in the composition.

### Antioxidants

Compositions of the present invention may include antioxidants to prevent or interrupt continuous oxidation that may initiate within the composition. Notable examples include sterically hindered phenolic antioxidants such as Pentaerythrityl Tetra-di-t-butyl Hydroxyhydrocinnamate available as TINOGARD TT and Octadecyl Di-t-butyl-4-hydroxyhydrocinnamate TINOGARD TS, both available from BASF. The concentration of antioxidant in the composition may be, for example, from about 0.05%, 0.1% or 0.15% to about 0.15%, 0.2%, 0.25% or 0.5% by weight in the composition.

### UV-Stabilizers

Compositions of the present invention, in certain embodiments, may include one or more organic ultraviolet (UV) stabilizers (also referred to herein as stabilizing compounds). By ultraviolet stabilizers it is meant organic ultraviolet compounds that are capable of protecting the composition from potentially deleterious effects of UV radiation through absorption of ultraviolet radiation or through excited state quenching. By “organic” it is meant compounds consisting substantially, predominantly or entirely of carbon, hydrogen, oxygen, and/or nitrogen atoms.

The UV stabilizers may include organic UV-filters commonly used in cosmetic sunscreens to provide personal protection to skin. By way of non-limiting examples these may include p-aminobenzoic acid derivatives, anthranilates, benzophenones, camphor derivatives, cinnamic derivatives, dibenzoyl methanes, β,β-diphenylacrylate derivatives, salicylic derivatives, triazine derivatives, benzimidazole compounds, bis-benzoazolyl derivatives, methylene bis-(hydroxyphenylbenzotriazole) compounds, and the like.

Other UV-stabilizers include various compounds often used to protect polymeric or other substrates—such as hindered amine compounds and/or excited state quenching compounds such as piperidine compounds. Piperidine is an organic compound with the molecular formula (CH₂)₅NH. It is a heterocyclic amine which is a six-membered ring containing five methylene bridges (—CH2—) and one amine bridge (—NH—). Piperidine compounds include derivatives of piperidine.

Non-limiting examples of hindered amines and piperidine compounds include poly[[6-[(1,1,3,3-tetramethylbutyl)amino]-1,3,5-triazine-2,4-diyl][(2,2,6,6-tetramethyl-4-piperidinyl) imino]-1,6 hexanediyl[(2,2,6,6-tetramethyl-4-piperidinyl)imino]] available as CHIMASSORB 944FDL from BASF; and Butanedioic acid, dimethylester, polymer with 4-hydroxy-2,2,6,6-tetramethyl-1-piperidine ethanol; available as TINUVIN 622LD, and Poly-{[6-[(1,1,3,3-tetramethylbutyl)-imino]-1,3,5-triazine-2,4-diyl][2-(2,2,6,6-tetramethylpiperidyl)- amino]-hexamethylene-[4-(2,2,6,6-tetramethylpiperidyl)-imino]} and Poly-(N-beta-hydroxyethyl-2,2,6,6-tetramethyl-4-hydroxy-piperidyl succinate) available as TINUVIN 783FDL from BASF.

Another derivative of piperidine is piperidinol, a piperidine compound which has at least one hydroxy (OH) substitution, and can contain up to 5 hydroxy substitutions such as, for example, 2 to 3 hydroxy substitutions, at carbon positions on the ring. Piperidinol can also be substituted with non-hydroxyl group substitutions at one or more carbon positions on the ring, if desired, and up to two such substitutions can occur at each ring position, if desired. Such additional non-hydroxyl group substitutions at one or more ring positions can include, but are not limited to, one or two substituted or unsubstituted, linear or branched, alkyl groups, preferably containing 1-8 carbon atoms, preferably 1-6 carbon atoms, preferably 1-3 carbon atoms, and preferably 1-2 carbon atoms.

Piperidinol can be in nonionic form, or in the form of a salt. Suitable salt forms of the piperidinol compound of the present invention include but are not limited to alkali metal salts (e.g., Na, K), alkaline earth salts (e.g., magnesium), and carboxylate salts such as salts of alpha hydroxy acids (e.g., lactates, citrates). One notable example of a piperidinol compound is tetramethylhydroxypiperidinol citrate. An example of a commercially-available product of this preferred piperidinol compound is Tinogard® Q available from BASF.

Other UV-stabilizers include benzotriazoles. Benzotriazoles are characterized by having two fused rings-of the aromatic compounds benzene and triazole. Benzotriazoles are heterocyclic compounds with the chemical formula C₆H₅N₃. Its five-membered ring contains three consecutive nitrogen atoms. Examples of benzotriazoles include benzotriazol dodedcyl p-cresol and [2-(2′-hydroxy-5′-methylphenyl) benzotriazole].

In particular embodiments, the organic UV-stabilizing compound is selected from a piperidinol compound, a benzotriazol compound, and combinations thereof. According to certain particular embodiments, the organic UV-stabilizing compound is selected from tetramethylhydroxypiperidinol citrate, benzotriazol dodedcyl p-cresol, and combinations thereof.

Preferably, the at least one film organic UV-stabilizing compound is present in the compositions of the present invention in an amount ranging from about 0.015% to about 1.5% by weight, preferably from about 0.02% to about 1% by weight, preferably from about 0.02% to about 0.5%, preferably from about 0.02% to about 0.2%, and preferably from about 0.02% to about 0.1% by weight with respect to the weight of the composition, including all ranges and subranges therebetween such as, for example, 0.02% to 0.08%, 0.025% to 0.1%, 0.05% to 0.2%, etc.

### C1-C4 Monoalcohols and C1-C4 Monoacetates

According to certain embodiments, the compositions are substantially free of either or both of C1-C4 monoalcohols and C1-C4 monoacetates. C1-C4 monoalcohols include for example isopropanol and ethanol. C1-C4 monoacetates include for example ethyl acetate and butyl acetate.

### Polymers

The cosmetic composition may include various polymers, such as polymers that may be readily solubilized or dispersed in the fatty phase. The polymers may include, for example C10-allylic polymers, acrylate polymers, polybutenes, polymeric elastomers, and the like. The total amount of polymers in the formulation may be from about 1% to about 40% by weight.

The cosmetic composition may include a C10-C40 alkyl acrylate polymer. The C10-C40 alkyl acrylate polymer may be a homopolymer. The C10-C40 alkyl acrylate polymer may be a copolymer. Non-limiting examples of such polymers include Acrylates/C12-22 Alkyl Methacrylate Copolymer, Acrylates/C12-22 Alkyl Methacrylate Copolymer, C12-22 Alkyl Acrylates/Hydroxyethylacrylate Copolymer, and/or acrylates/C10-C30 alkyl acrylate crosspolymers. In some embodiments, the C10-C40 alkyl acrylate polymer may be, e.g., TEGO® SP-6 (Poly C10-30 Alkyl Acrylate), TEGO® SP 13-1 (Poly C10-30 Alkyl Acrylate), and/or TEGOR SP 13-6 (Poly C10-30 Alkyl Acrylate). In some embodiments, the C10-C40 alkyl acrylate polymer may be, e.g., a modified acrylate homopolymer, such as a stearyl-or behenyl-modified acrylate homopolymer

The C10-C40 alkyl acrylate polymer may be present in an amount no more than 10% by weight of the composition. The C10-C40 alkyl acrylate polymer may be present in an amount no more than 5% by weight of the composition. In some embodiments, the C10-C40 alkyl acrylate polymer may be present in an amount no more than 4% by weight of the composition. In some embodiments, the C10-C40 alkyl acrylate polymer may be present in an amount no more than 3% by weight of the composition. In some embodiments, the C10-C40 alkyl acrylate polymer may be present in an amount no more than 2.5% by weight of the composition. In some embodiments, the C10-C40 alkyl acrylate polymer may be present in an amount of at least 0.5% by weight of the composition. In some embodiments, the C10-C40 alkyl acrylate polymer may be present in an amount of at least 1% by weight of the composition. In some embodiments, the C10-C40 alkyl acrylate polymer may be present in an amount of at least 1.5% by weight of the composition.

The cosmetic composition may include an allylic polymer. The term “allylic,” as used herein, describes an alkenyl group wherein a carbon-carbon double bond is connected to a methylene group, and the methylene group represents the point of attachment to the rest of the molecule. Non-limiting example of allylic polymers include allyl stearate/vinyl acetate (VA) copolymer, allyl 2,2-dimethylpentanoate/vinyl laurate, allyl dimethylpropionate/vinyl stearate, and allyl propionate/allyl stearate.

The allylic polymer may be present in an amount no more than 10% by weight of the composition. The allylic polymer may be present in an amount no more than 5% by weight of the composition. In some embodiments, the allylic polymer may be present in an amount no more than 4% by weight of the composition. In some embodiments, the allylic polymer may be present in an amount no more than 3% by weight of the composition. In some embodiments, the allylic polymer may be present in an amount no more than 2.5% by weight of the composition. In some embodiments, the allylic polymer may be present in an amount of at least 0.5% by weight of the composition. In some embodiments, the allylic polymer may be present in an amount of at least 1% by weight of the composition. In some embodiments, the allylic polymer may be present in an amount of at least 1.5% by weight of the composition.

### Polybutene

The composition may include a polybutene, such as those polybutenes used in cosmetic formulations. The polybutene may have a molecular weight (Mn) from about 500 to about 2000 daltons.

### Polymeric Elastomer

The composition may include a polymeric elastomer.

The polymeric elastomer may be a styrenic block copolymer elastomer.

The styrenic block copolymers of the present invention are characterized by the presence of at least one “hard” segment, and at least one “soft” segment. Aside from their compositional nature, the hard and soft segments of the block copolymers of the present invention may be defined in terms of their respective glass transition temperatures, T_{g}. The hard segment may have a T_{g} of 50° C. or more, whereas the soft segment may have a T_{g} of 20° C. or less. The Tg for the hard block can range from 50° C. to 150° C. The T_{g} for the soft block can range from −150° C. to 20° C.

Block copolymers useful in compositions of the present invention may be thermoplastic elastomers. The hard segments of the thermoplastic elastomer typically comprise vinyl monomers in varying amounts. Examples of suitable vinyl monomers include, but are not limited to, styrene as well as other optional monomers including methacrylate, acrylate, vinyl ester, vinyl ether, vinyl acetate, and the like. At least one hard segment should include styrene.

The soft segments of the thermoplastic elastomer comprise olefin polymers and/or copolymers which may be saturated, unsaturated, or combinations thereof. Suitable olefin copolymers may include, but are not limited to, ethylene/propylene copolymers, ethylene/butylene copolymers, propylene/butylene copolymers, polybutylene, polyisoprene, polymers of hydrogenated butanes and isoprenes, and mixtures thereof.

Thermoplastic elastomers useful in the present invention are block copolymers, e.g., di-block, tri-block, multi-block, radial and star block copolymers, and mixtures and blends thereof. A di-block thermoplastic elastomer is usually defined as an A-B type or a hard segment (A) followed by a soft segment (B) in sequence. A tri-block is usually defined as an A-B-A type copolymer or a ratio of one hard, one soft, and one hard segment. Multi-block or radial block or star block thermoplastic elastomers usually contain any combination of hard and soft segments, provided that the elastomers possess both hard and soft characteristics.

In some embodiments, the thermoplastic elastomer of the present invention may be chosen from the class of KRATON rubbers (Kraton Corporation of Houston, Tex.) or from similar thermoplastic elastomers. KRATON rubbers are thermoplastic elastomers in which the polymer chains comprise a di-block, tri-block, multi-block or radial or star block configuration or numerous mixtures thereof. The KRATON tri-block rubbers have polystyrene (hard) segments on each end of a rubber (soft) segment, while the KRATON di-block rubbers have a polystyrene (hard) segment attached to a rubber (soft) segment. The KRATON radial or star configuration may be a four-point or other multipoint star made of rubber with a polystyrene segment attached to each end of a rubber segment. The configuration of each of the KRATON rubbers forms separate polystyrene and rubber domains.

Each molecule of KRATON rubber is said to comprise block segments of styrene monomer units and rubber monomer and/or co-monomer units. The most common structure for the KRATON triblock copolymer is the linear A-B-A block type styrene-butadiene-styrene, styrene-isoprene-styrene, styrene-ethylenepropylene-styrene, or styrene-ethylene-butylene-styrene. The KRATON di-block is preferably the AB block type such as styrene-ethylene-propylene, styrene-ethylene-butylene, styrene-butadiene, or styrene-isoprene. The KRATON rubber configuration is well known in the art and any block copolymer elastomer with a similar configuration is within the practice of the invention. Other block copolymers are sold under the tradename Septon (which represent elastomers known as SEEPS, sold by Kurary, Co., Ltd) and those sold by ExxonMobil Chemical under the tradename VECTOR.

Other thermoplastic elastomers useful in the present invention may include those block copolymer elastomers comprising a styrene-butylene/ethylene-styrene copolymer (tri-block), an ethylene/propylene-styrene copolymer (radial or star block) or a mixture or blend of the two. (Some manufacturers refer to block copolymers as hydrogenated block copolymers, e.g. hydrogenated styrene-butylene/ethylene-styrene copolymer (tri-block)).

Compositions may include at least one block copolymer, e.g, diblock, triblock, multiblock or radial block copolymers, and mixtures thereof. The at least one block copolymer may include at least one styrene block and may further comprise at least one block comprising units chosen from butadiene, ethylene, propylene, butylene and isoprene or a mixture thereof.

Diblock copolymers that may be mentioned include but are not limited to styrene/ethylene-propylene copolymers (comprising a styrene block and a block obtained from ethylene and propylene), styrene/ethylene-butylene copolymers, styrene-ethylene/butadiene copolymers, styrene/butadiene copolymers and styrene/isoprene copolymers.

In certain notable embodiments, the block copolymer includes styrene blocks and one or more blocks selected from: butadiene blocks, isoprene blocks, and ethylene-butylene blocks. In other embodiments, the block copolymer includes (1) styrene blocks and butadiene blocks; or (2) styrene and ethylene-butadiene blocks; or (3) styrene and isoprene blocks.

Triblock copolymers that may be mentioned include but are not limited to styrene/ethylene-propylene/styrene copolymers, styrene/ethylene-butylene/styrene copolymers, styrene/ethylene-butadiene/styrene copolymers, styrene/isoprene/styrene copolymers and styrene/butadiene/styrene copolymers.

In some embodiments, the composition may include a mixture of a diblock copolymer and a triblock copolymer. copolymer. According to at least one embodiment, the diblock copolymer and the triblock copolymer may be chosen from block copolymers comprising at least one styrene block and at least one block comprising units chosen from butadiene, ethylene, propylene, butylene and isoprene. In one embodiment, the block copolymer has from about 50% to about 90% triblock and from about 10% to about 50% diblock.

In some embodiments, the composition may include a butylene/ethylene/styrene copolymer or an ethylene/propylene/styrene copolymer. In some embodiments, the composition may include both a butylene/ethylene/styrene copolymer and an ethylene/propylene/styrene copolymer.

The polymeric elastomer may be a silicone elastomer.

The silicone elastomer may be a non-emulsifying silicone elastomer. The term “non-emulsifying” defines organopolysiloxane elastomers not containing any hydrophilic chains, and in particular not containing any polyoxyalkylene units (especially polyoxyethylene or polyoxypropylene) or any polyglyceryl units. Thus, according to one particular mode of the invention, the composition comprises an organopolysiloxane elastomer free of polyoxyalkylene units and of polyglyceryl units.

Non-limiting examples of the silicone elastomer include Dimethicone Crosspolymer (INCI name), Vinyl Dimethicone Crosspolymer (INCI name), Dimethicone/Vinyl Dimethicone Crosspolymer (INCI name), Dimethicone Crosspolymer-3 (INCI name).

The silicone elastomer may be a self-emulsifying silicone elastomer. Non-limiting examples of self-emulsifying silicone elastomers include, but are not limited to, substituted or unsubstituted dimethicone/copolyol crosspolymer, dimethicone (and) dimethicone/vinyldimethicone crosspolymer, dimethicone and dimethicone/PEG-10/15 crosspolymers, substituted or unsubstituted dimethicone/polyglyceral crosspolymer, dimethicone and dimethicone/polyglycerin-3 crosspolymer.

The polymeric elastomer may be present in a total amount of not more than 3% by weight of the composition. The polymeric elastomer may be present in a total amount of not more than 2% by weight of the composition. The polymeric elastomer may be present in a total amount of not more than 1% by weight of the composition. The polymeric elastomer may be present in a total amount of not more than 0.5% by weight of the composition. In some embodiments, the composition may be free, or substantially free, of styrenic block copolymers. In some embodiments, the composition may be free, or substantially free, of non-emulsifying silicone elastomers. In some embodiments, the composition may be free, or substantially free, of self-emulsifying silicone elastomers.

### Dispersing Agent

The composition may include a dispersing agent. The dispersing agent serves to protect the dispersed particles against agglomeration or flocculation thereof. The dispersing agent may physically or chemically attach to the surface of the pigments. These dispersants may also contain at least one functional group that is compatible with or soluble in the continuous medium. In particular, 12-hydroxystearic acid esters and in particular 08 to 020 fatty acid esters of polyols such as glycerol or diglycerol may be used, such as poly (l2-hydroxystearic acid) stearate with a molecular weight of about 750 g/mol, such as the product sold under the name Solsperse 21 000 by the company Avecia, polyglyceryl-2 dipolyhydroxystearate (CTFA name) sold under the reference Dehymyls PGPH by the company Henkel,, or polyhydroxystearic acid such as the product sold under the reference Arlacel P100 by the company Uniqema, and mixtures thereof.

The dispersing agent may be present in a total amount of not more than 10% by weight of the composition. The dispersing agent may be present in a total amount of not more than 9% by weight of the composition. The dispersing agent may be present in a total amount of not more than 8% by weight of the composition. The dispersing agent may be present in a total amount of not more than 7% by weight of the composition. The dispersing agent may be present in a total amount of not more than 6% by weight of the composition. The dispersing agent may be present in a total amount of at least 1% by weight of the composition. The dispersing agent may be present in a total amount of at least 2% by weight of the composition. The dispersing agent may be present in a total amount of at least 3% by weight of the composition. The dispersing agent may be present in a total amount of at least 4% by weight of the composition. The dispersing agent may be present in a total amount of at least 5% by weight of the composition.

### Hydrocarbon-Based Resin

The composition may include a hydrocarbon-based resin. The hydrocarbon-based resin may be a hydrocarbon-based film former.

The composition according to the invention may include at least one hydrocarbon-based resin with a number-average molecular weight of less than or equal to 10 000 g/mol. The number-average molecular weight may range from 250 g/mol to 10 000 g/mol, preferably from 250 g/mol to 5000 g/mol, more preferentially from 250 g/mol to 2000 g/mol, or even from 250 g/mol to 1000 g/mol. The number-average molecular weights (Mn) may be determined by gel permeation liquid chromatography (THF solvent, calibration curve established with linear polystyrene standards, refractometric detector).

The softening point of the resin ranges from 70 to 130° C., more particularly from 80 to 120° C. and preferably from 90 to 110° C. (standard ASTM E 28).

The hydrocarbon-based resin may be an aliphatic hydrocarbon-based resin. Non-limiting examples of aliphatic hydrocarbon-based resins include:
hydrogenated indene/methylstyrene/styrene copolymers, for example sold under the name Regalite by the company Eastman Chemical, in particular Regalite R1090, Regalite R1100, Regalite S1100 and Regalite S5100, or under the name Arkon P-90, Arkon P-100, and Arkon P-115 by the company Arakawa;
aliphatic pentanediene resins especially such as those derived from the majority polymerization of the 1,3-pentanediene (trans- or cis-piperylene) monomer and of minority monomers chosen from isoprene, butene, 2-methyl-2-butene, pentene and 1,4-pentanediene, and mixtures thereof. These resins may have a molecular weight ranging from 1000 to 2500 g/mol. Such 1,3-pentanediene resins are sold, for example, under the references Piccotac 95 by the company Eastman Chemical, Escorez 1102, Escorez 1304, Escorez 1310LC and Escorez 1315 by the company Exxon Chemicals, or Wingtack 95 by the company Cray Valley;
diene resins of cyclopentanediene dimers, for instance those derived from the polymerization of dicyclopentanediene, methyldicyclopentanediene and other pentanediene dimers, and mixtures thereof. These resins generally have a molecular weight ranging from 500 to 800 g/mol, for instance those sold under the reference Escorez 5380, Escorez 5300, Escorez 5400, Escorez 5415 or Escorez 5490 by the company ExxonMobil Chem., and the resins Sukorez SU-90, Sukorez SU-100, Sukorez SU-110, Sukorez SU-100S, Sukorez SU-200, Sukorez SU-210, Sukorez SU-490 and Sukorez SU-400, by the company Kolon;
hydrogenated resins derived from the polymerization of pentanediene, such as, inter alia, those sold under the name Eastotac H-100E, Eastotac H-115E, Eastotac C-100L, Eastotac C-115L, Eastotac H-100L, Eastotac H-115L, Eastotac C-100R, Eastotac C-115R, Eastotac H-100R, Eastotac H-115R, Eastotac C-100W, Eastotac C-115W, Eastotac H-100W and Eastotac H-115W, by the company Eastman Chemical Co.;
and mixtures thereof.

One such resin may be, the hydrogenated indene/methylstyrene/styrene copolymer sold especially under the name Regalite® R1110 resin by the company Eastman Chemical, or the aliphatic 1,3-pentanediene resin sold under the name Piccotac® 1095 resin by the company Eastman Chemical.

Preferably, the content of resin according to the invention is from 2% to 20% by weight, more particularly from 2% to 18% by weight and preferably from 3% to 16% by weight relative to the total weight of the composition.

The hydrocarbon-based resin may be present in a total amount of not more than 6% by weight of the composition. The hydrocarbon-based resin may be present in a total amount of not more than 5% by weight of the composition. The hydrocarbon-based resin may be present in a total amount of not more than 4% by weight of the composition. The hydrocarbon-based resin may be present in a total amount of at least 1% by weight of the composition. The hydrocarbon-based resin may be present in a total amount of at least 2% by weight of the composition. The hydrocarbon-based resin may be present in a total amount of at least 3% by weight of the composition.

### Polyolefin

The composition may include a polyolefin. The polyolefin may be a polyolefin wax. As used herein, the term “wax” is intended to mean lipophilic compounds, which are solid at ambient temperature (20° C.) and at atmospheric pressure (760 mmHg), with a reversible solid/liquid change of state, which have a melting point of greater than or equal to 40° C., which may range up to 120° C. In some embodiments, the polyolefin waxes may have a melting point of at least about 80° C. In some embodiments, the polyolefin waxes may have a melting point of at least 80° C. The polyolefin waxes may be, e.g., homopolymers or copolymers of ethylene, of propene or butene, or even longer-chain a-olefins. In some embodiments, the polyolefin is polyethylene.

The polyolefin may be present in a total amount of not more than 5% by weight of the composition. The polyolefin may be present in a total amount of not more than 3% by weight of the composition. The polyolefin may be present in a total amount of not more than 2% by weight of the composition. The polyolefin may be present in a total amount of not more than 1.5% by weight of the composition. The polyolefin may be present in a total amount of not more than 1% by weight of the composition. The polyolefin may be present in a total amount of at least 0.1% by weight of the composition. The polyolefin may be present in a total amount of at least 0.25% by weight of the composition. The polyolefin may be present in a total amount of at least 0.5% by weight of the composition.

### Emulsifying Agent or Stabilizer

In certain embodiments of the invention it is desirable to include an emulsifying agent (“emulsifier”) or stabilizer. The emulsifying agent or stabilizer may be suitable to stabilize, for example, an emulsified internal phase (such as one including polyol) within the continuous fatty phase. The emulsifying agent or stabilizer may be present in a total amount of no more than 10% by weight of the composition. The emulsifying agent or stabilizer may be present in a total amount of no more than 8% by weight of the composition. In some embodiments, the emulsifying agent or stabilizer may be present in a total amount of no more than 4.5% by weight of the composition. In some embodiments, the emulsifying agent or stabilizer may be present in a total amount of no more than 4% by weight of the composition. In some embodiments, the emulsifying agent or stabilizer may be present in a total amount of no more than 3.5% by weight of the composition. In some embodiments, the emulsifying agent or stabilizer may be present in a total amount of at least 1% by weight of the composition. In some embodiments, the emulsifying agent or stabilizer may be present in a total amount of at least 1.5% by weight of the composition. In some embodiments, the emulsifying agent or stabilizer may be present in a total amount of at least 2% by weight of the composition. In some embodiments, the emulsifying agent or stabilizer may be present in a total amount of at least 2.5% by weight of the composition.

The emulsifying agent or stabilizer may comprise or consist of a fatty acid ester of a polyol. The fatty acid esters may be esters of isostearic acid and glycerol, such as for example polyglycerolated (4 moles) isostearate (INCI name: Polyglyceryl-4 Isostearate) sold under the name Isolan GI34® by Evonik, polyglycerolated (3 mol) diisostearate sold under the name Lameform TGI® by BASF; polyglycerolated distearate (2 mol) sold under the name Emalex PGSAR by Nihon emulsion; polyglycerolated (10 mol) monoisostearate sold under the name Nikkol Decaglyn 1-IS® by Nihon Surfactant (INCI name: Polyglyceryl-10 isostearate); and polyglyceryl-4 diisostearate/polyhydroxystearate/sebacate sold under the name Isolan GPS® by Evonik. In a preferred embodiment, the only emulsifying agent or stabilizer is polyglyceryl-4 diisostearate/polyhydroxystearate/sebacate.

### Organic Polar Solvent

In certain embodiments of the invention, it is desirable for the composition to include one or more organic polar solvents. The one or more organic polar solvents may be present in an internal phase such as emulsified within the continuous fatty phase. The organic polar solvent may be liquid at room temperature (25° C.) and at least partially water-miscible. Non-limiting examples include alkanols such as ethyl alcohol, isopropyl alcohol, aromatic alcohols such as benzyl alcohol and phenylethyl alcohol, or polyols or polyol ethers, for instance ethylene glycol monomethyl, monoethyl or monobutyl ether, propylene glycol or ethers thereof, for instance propylene glycol monomethyl ether, butylene glycol, dipropylene glycol, and also diethylene glycol alkyl ethers, for instance diethylene glycol monoethyl ether or monobutyl ether.

The polar solvent may comprise or consist of a polyol. Non-limiting examples of polyols include ethylene glycol, propylene glycol such as 1,2-propylene glycol and 1,3-propylene glycol, glycerol, pentaerythritol, trimethylolpropane, 1,4,6-octanetriol, butanediol, pentanediol, hexanediol, dodecanediol, octanediol, chloropentanediol, glycerol monoallyl ether, glycerol monoethyl ether, diethylene glycol, 2-ethylhexanediol-1,4, cyclohexanediol-1,4, 1,2,6-hexanetriol, neopental glycol, 1,3,5-hexanetriol, 1,3-bis-(2-hydroxyethoxy) propane and the like. In some embodiments, the polyol may comprise or consist of glycerin. The polyol may comprise or consist of glycerin and one or more glyceryl ethers or glyceryl esters. Non-limiting examples of glyceryl ethers include, e.g., alkyl glyceryl ethers such as isostearyl glyceryl ether, and ethylhexyl glycerin. Non-limiting examples of glyceryl esters includes glyceryl mono fatty acid esters such as glyceryl caprylate, glyceryl caprate, glyceryl laurate, glyceryl myristate, glyceryl stearate, glyceryl linoleate, glyceryl oleate, glyceryl isostearate, and glyceryl behenate.

In some embodiments, the polar solvent may be a water-soluble solvent. In some embodiments, the polar solvent may be a water-miscible solvent. In some embodiments, the polar solvent may include both a water-soluble solvent and a water-miscible solvent. As used herein, the term “water-soluble solvent” refers to compounds that are liquid at 25° C. and at atmospheric pressure (760 mmHg), and it has a solubility of at least 70% in water under these conditions. In some cases, the water-soluble solvent has a solubility of at least 80%, or at least 90% in water. As used herein, the term “water-miscible solvent” refers to compounds that are liquid at 25° C. and at atmospheric pressure (760 mmHg), and it has a solubility of at least 50% in water under these conditions. In some cases, the water-miscible solvent has a solubility of at least 60% in water under these conditions. In certain embodiments, the one or more polyols present in the composition have a molecular weight from 70 to 200 daltons and/or have from three to six carbon atoms.

The polar solvent may be present in a total amount of not more than 20% by weight of the composition. The polar solvent may be present in a total amount of not more than 15% by weight of the composition. The polar solvent may be present in a total amount of not more than 10% by weight of the composition. The polar solvent may be present in a total amount of not more than 9% by weight of the composition. The polar solvent may be present in a total amount of not more than 8% by weight of the composition. The polar solvent may be present in a total amount of not more than 7% by weight of the composition. The polar solvent may be present in a total amount of not more than 6% by weight of the composition. The polar solvent may be present in a total amount of at least 1% by weight of the composition. The polar solvent may be present in a total amount of at least 2% by weight of the composition. The polar solvent may be present in a total amount of at least 3% by weight of the composition. The polar solvent may be present in a total amount of at least 4% by weight of the composition. The polar solvent may be present in a total amount of at least 5% by weight of the composition.

### Other Ingredients

The composition may include a preservative. The preservative may include an organic acid preservative. Non-limiting examples of preservatives include benzoic acid and alkali metal and ammonium salts thereof (e.g. sodium benzoate), sorbic acid and alkali metal and ammonium salts thereof (e.g. potassium sorbate}, p-Anisic acid and alkali metal, and ammonium salts thereof, and salicylic acid and alkali metal and ammonium salts thereof, phenoxyethanol, benzyl alcohol, salicylic acid, sodium benzoate, caprylyl glycol, methyl paraben, propyl paraben, elhylhexylglycerin, 1,2-propanediol and any mixture thereof.

The preservative may be present in a total amount of no more than 2% by weight of the composition. The preservative may be present in a total amount of no more than 1.5% by weight of the composition. The preservative may be present in a total amount of no more than 1% by weight of the composition. The composition may be substantially free of preservatives.

The composition may include a skin care agent. The term “skin care agent” means an agent for enhancing the aesthetic appearance and/or comfort of the skin.

The skin care agent may be ceramide. Ceramides are a family of waxy lipid molecules that are composed of sphingosine and a fatty acid. Ceramides include ceramide 1, ceramide 2, ceramide 3, ceramide 4, ceramide 5, ceramide 1A, ceramide 6 II, ceramide AP, ceramide EOP, ceramide EOS, ceramide NP, ceramide NG, ceramide NS, ceramide AS, and ceramide NS dilaurate.

The composition may include a ceramide in a total amount of no more than 2% by weight of the composition. The composition may include a ceramide in a total amount of no more than 1.5% by weight of the composition. The composition may include a ceramide in a total amount of no more than 1% by weight of the composition. The composition may include a ceramide in a total amount of no more than 0.5% by weight of the composition. The composition may include a ceramide in a total amount of no more than 0.25% by weight of the composition.

The skin care agent may include a probiotic lysate. A probiotic is known, traditionally, as “microorganisms that, when administered in adequate amounts, confer a health benefit on the host.” Here, the composition use lysates of such microorganisms. A non-limiting example of such a lysate is bifida ferment lysate.

The skin care agent may include hyaluronic acid or a derivative thereof. In some embodiments, only a single hyaluronic acid or a derivative thereof is present. In some embodiments, a plurality of hyaluronic acid or a derivative thereof are present.

In the context of the present invention, the term “hyaluronic acid or a derivative thereof” covers the basic unit of hyaluronic acid which includes the smallest fraction of hyaluronic acid comprising a disaccharide dimer, namely D-glucuronic acid and N-acetylglucosamine.

The term “hyaluronic acid or a derivative thereof” also comprises, in the context of the present invention, the linear polymer comprising the polymeric unit described above, linked together in the chain via alternating beta(1,4) and beta(1,3) glycosidic linkages, having a molecular weight (MW) that can range between 380 and 13,000,000 daltons (Da). This molecular weight depends in large part on the source from which the hyaluronic acid is obtained and/or on the preparation methods.

The term “hyaluronic acid or a derivative thereof” also comprises, in the context of the present invention, the hyaluronic acid salts, and in particular the alkali metals salts such as the sodium salt and the potassium salt.

In the natural state, hyaluronic acid is present in pericellular gels, in the base substance of the connective tissues of vertebrate organs such as the dermis and epithelial tissues, and in particular in the epidermis, in the synovial fluid of the joints, in the vitreous humor, in the human umbilical cord and in the crista galli apophysis.

Thus, the term “hyaluronic acid or a derivative thereof” comprises all the fractions or subunits of hyaluronic acid having a molecular weight in particular within the molecular weight range recalled above.

According to a preferred embodiment of the invention the hyaluronic acid fractions suitable for the use covered by the present invention have a molecular weight of between 50,000 and 5,000,000, in particular between 100,000 and 5,000,000, especially between 400,000 and 5,000,000 Da. In this case, the term used is high-molecular-weight hyaluronic acid.

Alternatively, the hyaluronic acid fractions that may also be suitable for the use in the present invention are chosen from those with a molecular weight of between 50,000 and 400,000 Da (intermediate-molecular-weight hyaluronic acid) and from those with a molecular weight of less than 50,000 Da (low-molecular-weight hyaluronic acid).

Finally, the term “hyaluronic acid or a derivative thereof” also comprises hyaluronic acid esters in particular those in which all or some of the carboxylic groups of the acid functions are esterified with oxyethylenated alkyls or alcohols, containing from 1 to 20 carbon atoms, in particular with a degree of substitution at the level of the D-glucuronic acid of the hyaluronic acid ranging from 0.5 to 50 percent. Mention may in particular be made of methyl, ethyl, n-propyl, n-pentyl, benzyl and dodecyl esters of hyaluronic acid.

In some embodiments, the hyaluronic acid or a derivative thereof may be present in a total amount that is no more than about 2% by weight of the composition. In some embodiments, the hyaluronic acid or a derivative thereof may be present in a total amount that is no more than about 1% by weight of the composition. In some embodiments, the hyaluronic acid or a derivative thereof may be present in a total amount that is no more than about 0.5% by weight of the composition. In some embodiments, the hyaluronic acid or a derivative thereof may be present in a total amount that is no more than about 0.25% by weight of the composition. In some embodiments, the hyaluronic acid or a derivative thereof may be present in a total amount that is no more than about 0.1% by weight of the composition. In some embodiments, the composition may be substantially free of hyaluronic acid or a derivative thereof.

The skin care agent may include at least one hydrophilic active agent chosen from C-glycosides or derivatives thereof of the following general formula (I):

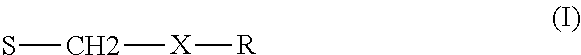

in which:R denotes an unsubstituted linear C₁-C₄and especially C₁-C₂ alkyl radical, in particular methyl; S represents a monosaccharide chosen from D-glucose, D-xylose, N-acetyl-D-glucosamine and L-fucose, and in particular D-xylose; X represents a group chosen from —CO—, —CH(OH)— and —CH(NH₂)— and preferentially a —CH(OH)— group; and also the cosmetically acceptable salts thereof, solvates thereof such as hydrates, and optical isomers thereof.

As non-limiting illustrations of C-glycoside derivatives of formula (I) that are more particularly suitable for the invention, mention may especially be made of the following compounds: C-beta-D-xylopyranoside-n-propan-2-one; C-alpha-D-xylopyranoside-n-propan-2-one; C-beta-D-xylopyranoside-2-hydroxypropane; C-alpha-D-xylopyranoside-2-hydroxypropane; 1-(C-beta-D-glucopyranosyl)-2-hydroxypropane; 1-(C-alpha-D-glucopyranosyl)-2-hydroxypropane; 1-(C-beta-D-glucopyranosyl)-2-aminopropane; 1-(C-alpha-D-glucopyranosyl)-2-aminopropane; 3′-(acetamido-C-beta-D-glucopyranosyl) propan-2′-one; 3′-(acetamido-C-alpha-D-glucopyranosyl)propan-2′-one; 1-(acetamido-C-beta-D-glucopyranosyl)-2-hydroxypropane; 1-(acetamido-C-beta-D-glucopyranosyl)-2-aminopropane; and also the cosmetically acceptable salts thereof, solvates thereof such as hydrates, and optical isomers thereof.

According to a particular embodiment, a C-glycoside of formula (I) that is suitable for the invention may advantageously be C-beta-D-xylopyranoside-2-hydroxypropane, the INCI name of which is Hydroxypropyl Tetrahydropyrantriol, sometimes referred to as proxylane.

The salts of the C-glycosides of formula (I) that are suitable for the invention may comprise conventional physiologically acceptable salts of these compounds, such as those formed from organic or inorganic acids. Examples that may be mentioned include the salts of mineral acids, such as sulfuric acid, hydrochloric acid, hydrobromic acid, hydriodic acid, phosphoric acid and boric acid. Mention may also be made of the salts of organic acids, which may comprise one or more carboxylic, sulfonic or phosphonic acid groups. They may be linear, branched or cyclic aliphatic acids, or alternatively aromatic acids. These acids may also comprise one or more heteroatoms chosen from O and N, for example in the form of hydroxyl groups. Mention may especially be made of propionic acid, acetic acid, terephthalic acid, citric acid and tartaric acid.

The solvates that are acceptable for the compounds described above comprise conventional solvates such as those formed during the final step of preparation of said compounds due to the presence of solvents. Examples that may be mentioned include solvates due to the presence of water or of linear or branched alcohols, such as ethanol or isopropanol.

According to one embodiment, the composition according to the invention comprises a C-glycoside in an amount of between 0.01% and 10% by weight of active material (C-glycoside) relative to the total weight of the composition. In some embodiments, the C-glycoside may be present in a total amount of less than 5% by weight. In some embodiments, the C-glycoside may be present in a total amount of less than 4% by weight. In some embodiments, the C-glycoside may be present in a total amount of less than 3% by weight. In some embodiments, the C-glycoside may be present in a total amount of less than 2% by weight. In some embodiments, the composition may be substantially free of the C-glycoside. In some embodiments, the composition may be free of the C-glycoside.

The skin care agent may include a peptide, such as a pentapeptide derivative (e.g., MATRIXIL® palmitoyl pentapeptide, synthetic peptides such as iamin, biopeptide CL, or a palmitoyloligopeptide, or peptides extracted from plants, such as the soybean hydrolysate, rice peptide, or peptide extracted from hazelnut.

According to one embodiment, the composition according to the invention comprises a peptide in an amount of between 0.01% and 10% by weight of a peptide relative to the total weight of the composition. In some embodiments, the peptide may be present in a total amount of less than 5% by weight. In some embodiments, the peptide may be present in a total amount of less than 4% by weight. In some embodiments, the peptide may be present in a total amount of less than 3% by weight. In some embodiments, the peptide may be present in a total amount of less than 2% by weight. In some embodiments, the composition may be substantially free of the peptide. In some embodiments, the composition may be free of the peptide.

In various embodiments, a method may be provided. The method may include providing a cosmetic composition as disclosed herein. The method may include applying the cosmetic composition to a lip or skin.

The composition may include one or more additional materials. The one or more additional materials may include any other cosmetically acceptable material. In some embodiments, the composition may include no more than 10% of the one or more additional materials. In some embodiments, the composition may include no more than 9% of the one or more additional materials. In some embodiments, the composition may include no more than 8% of the one or more additional materials. In some embodiments, the composition may include no more than 7% of the one or more additional materials. In some embodiments, the composition may include no more than 6% of the one or more additional materials. In some embodiments, the composition may include no more than 5% of the one or more additional materials. In some embodiments, the composition may include no more than 4% of the one or more additional materials. In some embodiments, the composition may include no more than 3% of the one or more additional materials. In some embodiments, the composition may include no more than 2% of the one or more additional materials. In some embodiments, the composition may be free, or substantially free, of the one or more additional materials.

### Filler

The one or more additional materials may include a filler. The filler may be present in a total amount of not more than 5% by weight of the composition. The filler may be present in a total amount of not more than 4% by weight of the composition. The filler may be present in a total amount of not more than 3% by weight of the composition. The filler may be present in a total amount of not more than 2% by weight of the composition. The filler may be present in a total amount of not more than 1% by weight of the composition.

The filler may be a mineral filler, an organic filler, or an optionally modified clay.

### Mineral Filler and/or Organic Filler

In some embodiments, the filler may be colorless or white solid particles of any form, which are in an insoluble form dispersed in the medium of the composition. These particles, of mineral or organic nature, give body or rigidity to the composition and/or softness and uniformity to the makeup.

The fillers used in the compositions according to the invention may have a lamellar, globular, spherical, platelet, or fibrous form or of any other form intermediate between these defined forms.

The fillers according to the invention may or may not be surface-coated, and in particular they may be surface-treated with silicones, amino acids, fluorinated derivatives or any other substance which promotes the dispersion and the compatibility of the filler in the composition.

Examples of mineral fillers that may be mentioned include talc, mica, silica, hollow silica microspheres, kaolin, calcium carbonate, magnesium carbonate, hydroxyapatite, boron nitride, glass or ceramic microcapsules, or composites of silica and of titanium dioxide, for instance the TSG series sold by Nippon Sheet Glass

Examples of organic fillers that may be mentioned include polyethylene powders, polymethyl methacrylate powders, polytetrafluoroethylene powders, acrylic acid copolymer powders (Polytrap from the company Dow Corning), lauroyl lysine, hollow polymer microspheres such as those of polyvinylidene chloride/acrylonitrile, for instance Expancel (Nobel Industrie), hexamethylene diisocyanate/trimethylol hexyllactone copolymer powder (Plastic Powder from Toshiki), silicone resin microbeads (for example Tospearl from Toshiba), synthetic or natural micronized waxes, metal soaps derived from organic carboxylic acids containing from 8 to 22 carbon atoms and preferably from 12 to 18 carbon atoms, for example zinc stearate, magnesium stearate, lithium stearate, zinc laurate or magnesium myristate, Polypore® L 200 (Chemdal Corporation), polyurethane powders, in particular powders of crosslinked polyurethane comprising a copolymer, said copolymer comprising trimethylol hexyllactone.

The mineral and/or organic filler may be present in a total amount of not more than 5% by weight of the composition. In some embodiments, the composition may be substantially free of any mineral and/or organic filler

### Optionally Modified Clay

The filler may be an optionally modified clay. Clays are silicates containing a cation which can be chosen from calcium, magnesium, aluminium, sodium, potassium or lithium cations, and their mixtures.

Mention may be made, as examples of such products, of clays of the family of the smectites, and also of the family of the vermiculites, stevensites or chlorites. These clays can be of natural or synthetic origin.

Preferably, use is made of organophilic clays, more particularly of modified clays, such as montmorillonite, bentonite, hectorite, attapulgite or sepiolite, and their mixtures. The clay is preferably a bentonite or a hectorite.

These clays are modified with a chemical compound chosen from quaternary amines, tertiary amines, amine acetates, imidazolines, amine soaps, fatty sulfates, alkylarylsulfonates or amine oxides, and their mixtures.

Mention may thus be made of hectorites modified by a quaternary amine, more specifically by a C₁₀ to C₂₂ fatty acid ammonium halide, such as a chloride, comprising or not comprising an aromatic group, such as hectorite modified by a distearyldimethylammonium halide, preferably a chloride, (CTFA name: Disteardimonium hectorite), such as, for example, those sold under the name Bentone 38V, Bentone 38V CG or Bentone EW CE by Elementis, or stearalkonium hectorites, such as in particular the product Bentone 27 V.

Mention may also be made of quaternium-18 bentonites, such as those sold, inter alia, under the names Bentone 34 by Elementis, Claytone 40, Tixogel VP by United Catalyst by Southern Clay; stearalkonium bentonites, such as those sold under the names Tixogel LG by United Catalyst and Claytone AF and Claytone APA by Southern Clay; or quaternium-18/benzalkonium bentonites, such as those sold under the name Claytone HT by Southern Clay.

The optionally modified clay may be present in a total amount of not more than 5% by weight of the composition. The optionally modified clay may be present in a total amount of not more than 4% by weight of the composition. The optionally modified clay may be present in a total amount of not more than 3% by weight of the composition. The optionally modified clay may be present in a total amount of not more than 2% by weight of the composition. The optionally modified clay may be present in a total amount of not more than 1% by weight of the composition.

### EXAMPLES

### Example I

Six compositions were made that included Red 28 pigment with and without various surface treatments. This includes one exemplary formula (Ex. 1) and five comparative formulas (Comp. 1, Comp. 2, and Comp. 3, Comp. 4, Comp. 5). See Table 1, below. The samples were prepared by first preparing 10% by weight pigment premixes by adding 10% by weight pigment to pentaerythrityl tetraisosterate. Separately, mixtures of additional oils and polymers, fatty compounds and other ingredients was blended at to about 90° C. After mixing and cooling these to room temperature, the appropriate pigment premixes was then added to form the compositions as detailed in Table 1.

The color change, ΔE was determined using the following Color Stability Test.

An Atlas Suntest CPS+ material testing weathering device (Ametek, Mount Prospect, IL) was used. Samples were placed in representative clear commercial lip gloss packaging. Duplicate samples were created for each testing parameter. One product (the control) was shielded from sun exposure in a closed/dark box and the other sample was used to run in the sun chamber to simulate UV and heat exposure with a controlled intensity and exposure time (765 W/m² for 24 hours).

The color stability in pack before and after sun test is assessed by using a DigiEye 700 mm cube instrument (Veri Vede Enderby, Leicester, UK). The pictures of the samples are taken in a controlled light box. the color shift analysis is performed with calibrated camera/software. For each composition, L*a*b* values were read and used to determine the ΔE as an indication of color shifting. A lower ΔE means there has been less color shift, while higher ΔE values indicate a higher level of color shift. Control (non UV exposed sample) were compared to the ΔE for the sample subjected to sun testing, thereby determining the amount of fading for each composition resulting from the sun testing.

The measured color change, ΔE is also shown in Table 1, as is the calculated percent improvement (%) over untreated pigment with no UV-stabilizers (“u, ns”), Comp. 5.

$$\% Improvement ( versus untreated ) = ( Δ {E}_{u , ns} - Δ {E}_{sample} ) / Δ {E}_{u , ns}$$

For the calculations ΔE_{u,ns} is 33.66.

TABLE 1
(Formulas)

| | Ex. 1 | Comp. 1 | Comp. 2 | Comp. 3 | Comp. 4 | Comp. 5 |
|---|---|---|---|---|---|---|
| Material | (% w/w) | (% w/w) | (% w/w) | (% w/w) | (% w/w) | (% w/w) |
| Fatty | 50% | 50% | 50% | 50% | 50% | 50% |
| Compounds | | | | | | |
| Polymers | 25% | 25% | 25% | 25% | 25% | 25% |
| Glycerin | 5% | 5% | 5% | 5% | 5% | 5% |
| Emulsifier | 3% | 3% | 3% | 3% | 3% | 3% |
| Active | 1% | 1% | 1% | 1% | 1% | 1% |
| Ingredients | | | | | | |
| Clay | 1% | 1% | 1% | 1% | 1% | 1% |
| Other | 15% | 15% | 15% | 15% | 15% | 15% |
| Ingredients | | | | | | |
| Pigment 1 | 0.01% | | | | | |
| Pigment 2 | | 0.01% | | | | |
| Pigment 3 | | | 0.01% | | | |
| Pigment 4 | | | | 0.01% | | |
| Pigment 5 | | | | | 0.01% | |
| Pigment 6 | | | | | | 0.01% |
| ΔEₛₐₘₚₗₑ | 19.08 | 22.75 | 26.12 | 23.34 | 30.22 | 33.66 |
| ΔE_{u, ns} | 33.66 | 33.66 | 33.66 | 33.66 | 33.66 | 33.66 |
| % Improvement | 43.3% | 32.4% | 22.4% | 30.7% | 10.2% | — |
| (vs untreated) | | | | | | |

The following colorants were used and are shown in the various tables: Pigment 1: Red 28 Lake treated with aloe; Pigment 2: Red 28 Lake treated with isopropyl titanium tristearate and triethoxysilylethyl polydimethylsiloxyethyl dimethicone, Pigment 3: Red 28 lake treated with isopropyl titanium triisostearate (and) bis-ppg-15 dimethicone/ipdi copolymer (and) peg-2 soyamine polymer with isophorone diisocyanate; Pigment 4: Red 28 Lake treated with triethoxycaprylyl silane; Pigment 5: Red 28 Lake treated with avocado oil; and Pigment 6: Red 28 Lake, untreated.

It can be seen that the aloe treated pigment performs substantially better than untreated pigment as well as compared to alternative surface treatments.

### Example II

Additionally, other compositions were made that included Red 28 pigment with and without various surface treatments, and further included UV stabilizer. The UV-stabilizer included tris (tetramethylhydroxypiperidonol) citrate (TINOGARD Q, also from BASF). The compositions are shown in Table 2 below. Components other than colorants were otherwise as shown in Table 1. The identity of pigments 1-6 are also described above. Pigment 5 was not tested (N/T).

The color change, ΔEₛₐₘₚₗₑ is also provided in Table 2, and also shown is the percent improvement (%) over untreated pigment
where % Improvement (versus untreated)=(ΔE_{u,ns}−ΔEₛₐₘₚₗₑ)/ΔE_{u,ns}
and AEu.ns is 33.66.

TABLE 2

| | Ex. 2 | Comp. 6 | Comp. 7 | Comp. 8 | Comp. 9 | Comp. 10 |
|---|---|---|---|---|---|---|
| Material | (% w/w) | (% w/w) | (% w/w) | (% w/w) | (% w/w) | (% w/w) |
| Pigment 1* | 0.01% | | | | | |
| Pigment 2* | | 0.01% | | | | |
| Pigment 3* | | | 0.01% | | | |
| Pigment 4* | | | | 0.01% | | |
| Pigment 5* | | | | | 0.01% | |
| Pigment 6* | | | | | | 0.01% |
| TINOGARD Q | 0.1% | 0.1% | 0.1% | 0.1% | 0.1% | 0.1% |
| (10% active) | | | | | | |
| ΔEₛₐₘₚₗₑ | 14.87 | 26.82 | 27.13 | 28.92 | N/T | 31.57 |
| ΔE_{u, ns} | 33.66 | 33.66 | 33.66 | 33.66 | 33.66 | 33.66 |
| % Improvement | 55.8% | 20.3% | 19.4% | 14.1% | — | 6.2% |
| (vs Untreated) | | | | | | |
| | Ex. 3 | Comp. 11 | Comp. 12 | Comp. 13 | Comp. 14 | Comp. 15 |
| | (% w/w) | (% w/w) | (% w/w) | (% w/w) | (% w/w) | (% w/w) |
| Pigment 1 | 0.01% | | | | | |
| Pigment 2 | | 0.01% | | | | |
| Pigment 3 | | | 0.01% | | | |
| Pigment 4 | | | | 0.01% | | |
| Pigment 5 | | | | | 0.01% | |
| Pigment 6 | | | | | | 0.01% |
| TINOGARD Q | 0.2% | 0.2% | 0.2% | 0.2% | 0.2% | 0.2% |
| (10% active) | | | | | | |
| ΔEₛₐₘₚₗₑ | 19.48 | 27.12 | 26.86 | 28.51 | N/T | 27.39 |
| ΔE_{u, ns} | 33.66 | 33.66 | 33.66 | 33.66 | 33.66 | 33.66 |
| % Improvement | 42.1% | 19.4% | 20.2% | 15.3% | — | 18.6% |
| (vs Untreated) | | | | | | |

It can be seen that for Example compositions Ex. 2-3 the color change is greatly reduced compared to the comparative examples and the % improvement vs a similar formula without surface treatment or UV-stabilizer for the aloe treated colorant is more than double the other colorants that were tested.

### Example III

Additionally, other compositions were made that included Red 28 pigment with and without various surface treatments, and further included various UV stabilizers. The UV-stabilizers included both tris (tetramethylhydroxypiperidonol) citrate (TINOGARD Q, also from BASF) and benzotriazol dodedcyl p-cresol (TINOGARD TL from BASF). The compositions are shown in Table 3 below. Components other than colorants were otherwise as shown in Table 1. The identity of pigments 1-6 are also described above.

The color change, ΔEₛₐₘₚₗₑ is also provided in Table 3, and also shown is the percent improvement (%) over untreated pigment
where % Improvement (versus untreated)=(ΔE_{u,ns}−ΔEₛₐₘₚₗₑ)/ΔE_{u,ns}
and ΔE_{u,ns} is 33.66.

TABLE 3

| | Ex. 4 | Comp. 16 | Comp. 17 | Comp. 18 | Comp. 19 | Comp. 20 |
|---|---|---|---|---|---|---|
| Material | (% w/w) | (% w/w) | (% w/w) | (% w/w) | (% w/w) | (% w/w) |
| Pigment 1 | 0.01% | | | | | |
| Pigment 2 | | 0.01% | | | | |
| Pigment 3 | | | 0.01% | | | |
| Pigment 4 | | | | 0.01% | | |
| Pigment 5 | | | | | 0.01% | |
| Pigment 6 | | | | | | 0.01% |
| TINOGARD Q | 0.1% | 0.1% | 0.1% | 0.1% | 0.1% | 0.1% |
| (10% active) | | | | | | |
| TINOGARD TL | 0.1% | 0.1% | 0.1% | 0.1% | 0.1% | 0.1% |
| ΔEₛₐₘₚₗₑ | 12.03 | 27.68 | 33.2 | 35.8 | 25.5 | 33.94 |
| ΔE_{u, ns} | 33.66 | 33.66 | 33.66 | 33.66 | 33.66 | 33.66 |
| % Improvement | 64.2% | 17.7% | 1.37% | −6.35% | 24.2% | −0.08% |
| (vs Untreated) | | | | | | |

It can again be seen that for Example compositions Ex. 4 the color change the color change is greatly reduced compared to the comparative examples and the % improvement vs a similar formula without surface treatment or UV-stabilizer for the aloe treated colorant is more than double the other colorants that were tested.

### Example IV

Additionally, other compositions were made that included Red 28 pigment with and without various surface treatments, and further included various UV stabilizers. The UV-stabilizers included only benzotriazol dodedcyl p-cresol (TINOGARD TL from BASF). The compositions are shown in Table 4 below. Components other than colorants were otherwise as shown in Table 1. The identity of pigments 1-6 are also described above.

The color change, AEsample is also provided in Table 4, and also shown is the percent improvement (%) over untreated pigment
where % Improvement (versus untreated)=(ΔE_{u,ns}−ΔEₛₐₘₚₗₑ)/ΔE_{u,ns}
and ΔE_{u,ns} is 33.66.

TABLE 4

| | Ex. 5 | Comp. 21 | Comp. 22 | Comp. 23 | Comp. 24 | Comp. 25 |
|---|---|---|---|---|---|---|
| Material | (% w/w) | (% w/w) | (% w/w) | (% w/w) | (% w/w) | (% w/w) |
| Pigment 1 | 0.01% | | | | | |
| Pigment 2 | | 0.01% | | | | |
| Pigment 3 | | | 0.01% | | | |
| Pigment 4 | | | | 0.01% | | |
| Pigment 5 | | | | | 0.01% | |
| Pigment 6 | | | | | | 0.01% |
| TINOGARD TL | 0.1% | 0.1% | 0.1% | 0.1% | 0.1% | 0.1% |
| ΔEₛₐₘₚₗₑ | 15.93 | 26.1 | 24.79 | 25.54 | 32.14 | 29.94 |
| ΔE_{u, ns} | 33.66 | 33.66 | 33.66 | 33.66 | 33.66 | 33.66 |
| % Improvement | 52.7% | 22.5% | 26.3% | 24.1% | 4.52% | 11.1% |
| (vs Untreated) | | | | | | |
| | Ex. 6 | Comp. 26 | Comp. 27 | Comp. 28 | Comp. 29 | Comp. 30 |
| | (% w/w) | (% w/w) | (% w/w) | (% w/w) | (% w/w) | (% w/w) |
| TINOGARD TL | 0.2% | 0.2% | 0.2% | 0.2% | 0.2% | 0.2% |
| ΔEₛₐₘₚₗₑ | 10.42 | 22.67 | 26.29 | 24.65 | 28.83 | 26.78 |
| ΔE_{u, ns} | 33.66 | 33.66 | 33.66 | 33.66 | 33.66 | 33.66 |
| % Improvement | 69.0% | 32.6% | 21.9% | 26.8% | 14.3% | 20.4% |
| (vs Untreated) | | | | | | |

It can again be seen that for Example compositions Ex. 5-6 the color change the color change is greatly reduced compared to the comparative examples and the % improvement vs a similar formula without surface treatment or UV-stabilizer for the aloe treated colorant is about double or more compared with the other colorants that were tested.

Those skilled in the art will recognize or be able to ascertain using no more than routine experimentation many equivalents to the specific embodiments of the invention described herein. Such equivalents are intended to be encompassed by the following claims.

What is claimed is:

1. A cosmetic composition, comprising:
a continuous fatty phase comprising one or more fatty substances;
a colorant dispersed in the fatty phase, wherein the colorant comprises
a substrate;
at least one dye; and
a surface treatment, wherein the surface treatment comprises aloe.

2. The cosmetic composition of claim 1, wherein the cosmetic composition is liquid.

3. The cosmetic composition of claim 1, wherein the colorant is insoluble in the continuous fatty phase.

4. The cosmetic composition of claim 1, wherein the at least one dye comprises at least a xanthene dye.

5. The cosmetic composition of claim 4, wherein the substrate comprises at least one metal salt.

6. The cosmetic composition of claim 4, wherein the substrate comprises an aluminum salt and the xanthene dye is disodium 2′,4′,5′,7′-tetrabromo-4,5,6,7-tetrachlorofluorescein.

7. The cosmetic composition of claim 1, wherein the colorant is present in a concentration by weight in the cosmetic composition from about 0.01% to about 10%.

8. The cosmetic composition of claim 1, wherein the surface treatment is a hydrophobic surface treatment and further comprises one or more vegetable fatty compounds.

9. The cosmetic composition of claim 1, wherein the aloe and the substrate are each is present in a respective concentration by weight in the cosmetic compositin and wherein the concentration by weight of the substrate in the cosmetic composition is greater than the concentration by weight of the aloe in the cosmetic composition.

10. The cosmetic composition of claim 1, wherein the fatty phase is an external fatty phase and the cosmetic composition comprises an internal phase comprising one or more polyols within the external fatty phase.

11. The cosmetic composition of claim 10, wherein the cosmetic composition further comprises a fatty acid ester of a polyol.

12. The cosmetic composition of claim 1, wherein the cosmetic composition is substantially free of C1-C4 monoalcohols and C1-C4 monoacetates.

13. The cosmetic composition of claim 1 further comprising a UV-stabilizing compound.

14. The cosmetic composition of claim 1 further comprising a UV-stabilizing compound selected from a piperidinol, a benzotriazole, and combinations thereof.

15. The cosmetic composition of claim 1 wherein the cosmetic composition comprises at least about 35% by weight of the one or more fatty substances.

16. A method, comprising:
providing a cosmetic composition according to claim 1; and
applying the cosmetic composition to lips, skin, or nails.
